(19) United States
(12) Patent Application Publication
(10) Pub. No.: US 2007/0264699 A1
(43) Pub. Date: Nov. 15, 2007

(54) Method for cyrstallizing human GSK3 and novel crystal structure thereof

(75) Inventors: Dirksen E. Bussiere, San Leandro, CA (US); Vincent P. Le, San Francisco, CA (US)

Correspondence Address: CHIRON CORPORATION;INTELLECTUAL PROPERTY - R440, PO BOX 8097, EMERYVILLE, CA, 94662-8097, US

(73) Assignee: Chiron Corporation, Emeryville, CA (US)

(21) Appl. No.: 11/824,457

(22) Filed: Jun. 29, 2007

Related U.S. Application Data
(62) Division of application No. 10/366,546, filed on Feb. 11, 2003, now abandoned.
(60) Provisional application No. 60/355,916, filed on Feb. 11, 2002.

(51) Int. Cl. C12N 9/12; C07K 1/107; C07K 14/00

(52) U.S. Cl. 435/194; 530/324; 530/402

(57) ABSTRACT

The invention provides the three-dimensional structure of a construct of human glycogen synthase kinase 3 (GSK3); crystals of a construct of human glycogen synthase kinase 3-β (GSK3-β) containing the protein's catalytic kinase domain; a method for crystallizing the protein construct to provide a GSK3 crystal sufficient for structure determination; and a method for using the GSK3 construct's three-dimensional structure for the identification of possible therapeutic compounds in the treatment of various disease conditions mediated by GSK3 activity.

### CROSS-REFERENCE TO RELATED APPLICATION

This application is a divisional of U.S. patent application Ser. No. 10/366,546, filed Feb. 11, 2003, which claims the benefit of U.S. Provisional Application No. 60/355,916, filed Feb. 11, 2002. Each application is incorporated herein by reference in its entirety.

### FIELD OF THE INVENTION

This invention relates to the three-dimensional structure of human glycogen synthase kinase 3 (GSK3); to crystals of a ternary complex of a GSK3 construct, adenosine diphosphate, and a phosphorylated peptide; to methods for forming crystals of the GSK3 ternary complex; to methods for determining the crystal structure of the GSK3 ternary complex; and to methods for using the three-dimensional structure of the GSK3 ternary complex to identify possible therapeutic compounds for the treatment of various disease conditions mediated by GSK3 activity.

### BACKGROUND OF THE INVENTION

Glycogen synthase kinase 3 (GSK3) is a serine/threonine kinase for which two isoforms, α and β, have been identified. Woodgett, Trends Biochem. Sci., 16:177-81 (1991). Both GSK3 isoforms are constitutively active in resting cells. GSK3 was originally identified as a kinase that inhibits glycogen synthase by direct phosphorylation. Upon insulin activation, GSK3 is inactivated, thereby allowing the activation of glycogen synthase and possibly other insulin-dependent events, such as glucose transport. Subsequently, it has been shown that GSK3 activity is also inactivated by other growth factors that, like insulin, signal through receptor tyrosine kinases (RTKs). Examples of such signaling molecules include IGF-1 and EGF Saito et al., Biochem. J. 303:27-31, 1994; Welsh et al., Biochem. J. 294:625-29, 1993; and Cross et al., Biochem. J. 303:21-26, 1994.

Agents that inhibit GSK3 activity are useful in the treatment of disorders that are mediated by GSK3 activity. In addition, inhibition of GSK3 mimics the activation of growth factor signaling pathways and consequently GSK3 inhibitors are useful in the treatment of diseases in which such pathways are insufficiently active. Examples of diseases that can be treated with GSK3 inhibitors include diabetes, Alzheimer's disease, CNS disorders such as bipolar disorder, and immune potentiation-related conditions, among others.

Because inhibitors of GSK3 are useful in the treatment of many diseases, the identification of new inhibitors of GSK3 would be highly desirable. The present invention provides a method for identifying possible therapeutic compounds for the treatment of various disease conditions mediated by GSK3 activity. The method of the present invention utilizes the three-dimensional structure of a GSK3 ternary complex that contains the protein's catalytic domain to identify possible therapeutic compounds and to optimize the structure of lead therapeutic compounds.

### SUMMARY OF THE INVENTION

In accordance with the present invention, the three-dimensional structure of ternary complex of a construct of human glycogen synthase kinase 3 (GSK3), adenosine diphosphate, and a phosphorylated peptide is provided.

In one aspect, the invention provides crystals of a ternary GSK3 complex including a construct of human glycogen synthase kinase 3-β (GSK3-β) containing the protein's catalytic kinase domain, adenosine diphosphate, and a phosphorylated peptide. In one embodiment, the crystal includes a GSK3 construct and a phosphorylated peptide. The GSK3 construct can have the amino acid sequence set forth in SEQ ID NO: 1 or an active mutant or variant thereof. The phosphorylated peptide can be a diphosphorylated polypeptide. The crystal can have the atomic coordinates set forth in Table 2.

In another aspect of the invention, a method for crystallizing the GSK3 ternary complex to provide a GSK3 crystal sufficient for structure determination is provided. In one embodiment, the method includes crystallizing a purified GSK3 protein to provide a crystallized GSK3 protein having biological activity, wherein the crystallized GSK3 protein comprises a GSK3 construct and a phosphorylated polypeptide and wherein the crystallized GSK3 protein is resolvable using x-ray crystallography to obtain x-ray patterns suitable for three-dimensional structure determination of the crystallized GSK3 protein. In one embodiment, crystallizing the GSK3 protein includes crystallizing by a hanging drop vapor diffusion method. The GSK3 construct can have the amino acid sequence set forth in SEQ ID NO: 1 or an active mutant or variant thereof. The phosphorylated peptide can be a diphosphorylated polypeptide. The crystal can have the atomic coordinates set forth in Table 2. A crystallized GSK3 protein provided by the method is also provided.

In a further aspect, a method for making a GSK3 protein complex is provided. In one embodiment, the method includes combining a polypeptide that is capable of being phosphorylated, adenosine triphosphate, a magnesium salt, and a GSK3 protein to provide a GSK3 protein complex comprising a phosphorylated polypeptide, adenosine diphosphate, and the GSK3 protein. In this embodiment, the polypeptide capable of being phosphorylated can be a monophosphorylated polypeptide. In another embodiment, the method includes combining a phosphorylated polypeptide, adenosine diphosphate, and a GSK3 protein to provide a GSK3 protein complex comprising a phosphorylated polypeptide, adenosine diphosphate, and the GSK3 protein. In this embodiment, the phosphorylated polypeptide can be a diphosphorylated polypeptide.

Crystals can be made from these GSK3 protein complexes by adding a precipitant to solutions containing these complexes. Suitable precipitants include polyethylene glycol and 2-methyl-2,4-pentanediol. The GSK3 protein can have the amino acid sequence set forth in SEQ ID NO: 1 or an active mutant or variant thereof. The crystal can have the atomic coordinates set forth in Table 2.

In yet another aspect of the invention, a method for making a GSK3 protein crystal that includes a potential GSK3 mediator is provided. In one embodiment, the method includes contacting a crystallized GSK3 protein with a potential GSK3 mediator. The crystallized GSK3 protein can include a GSK3 construct and a phosphorylated polypeptide. A crystal produced by the method is also provided.

In another aspect, the invention provides a method for providing an atomic model of a GSK3 protein. In one embodiment, the method includes the steps: (a) providing a computer readable medium having stored thereon atomic coordinate/x-ray diffraction data of a GSK3 protein in crystalline form, the data sufficient to model the three-dimensional structure of the GSK3 protein, and the GSK3 protein in crystalline form includes a GSK3 construct and a phosphorylated polypeptide; (b) analyzing the atomic coordinate/x-ray diffraction data from step (a) to provide data output defining an atomic model of the GSK3 protein; and (c) obtaining atomic model output data defining the three-dimensional structure of the GSK3 protein.

A computer readable medium having stored thereon atomic model data of the GSK3 protein produced by the method and a GSK3-β ligand corresponding to the physical model of the atomic model of the ligand model produced by the method are also provided.

In yet another aspect, a method is provided for using the GSK3 ternary complex's three-dimensional structure for the identification of possible therapeutic compounds in the treatment of various disease conditions mediated by GSK3 activity. In one embodiment, the invention provides a method for designing ligands that bind to a GSK3 protein, comprising using some or all of the atomic coordinates of the GSK3 complex. In one embodiment, the method includes the steps: (a) crystallizing a purified GSK3 protein to provide a crystallized GSK3 protein having biological activity, wherein the crystallized GSK3 protein comprises a GSK3 construct and a phosphorylated polypeptide; (b) resolving the structure of the crystallized GSK3 protein using x-ray crystallography to obtain data suitable for three-dimensional structure determination of the GSK3 protein; (c) applying the data generated from resolving the structure of the crystallized GSK3 protein to a computer algorithm to generate a model of the GSK3 protein suitable for use in designing ligands that will bind to the GSK3 protein active site; and (d) applying an iterative process whereby molecular structures are applied to the computer generated model to identify GSK3 binding ligands. The crystallized GSK3 protein can include the atomic coordinates set forth in Table 2. The GSK3 protein can include the amino acid sequence set forth in SEQ ID NO: 1 or an active mutant or variant thereof. A GSK binding ligand designed by the method is also provided.

In another aspect, the invention provides a method for identifying a GSK3 mediator by determining the binding interactions between a potential mediator and a GSK3 binding site, the binding site being defined by at least some of a GSK3 crystal's atomic coordinates. In one embodiment, the method includes the steps: (a) generating a binding cavity defined by the binding site on a computer screen; (b) generating compounds with their spatial structure; and (c) determining whether the compounds bind at the GSK3 binding site. The invention also provides a method for identifying a compound that mediates GSK3 activity. In one embodiment, the method includes the steps: (a) designing a potential mediator for GSK3 that will form non-covalent bonds with amino acids in the GSK3 binding site based on at least some of the GSK3 crystal's atomic coordinates; (b) obtaining the potential mediator; and (c) determining whether the potential mediator mediates the activity of GSK3. In another embodiment, the method includes the steps: (a) using a three-dimensional structure of GSK3 as defined by the GSK3 crystal's atomic coordinates to design or select the potential mediator; (b) obtaining the potential mediator; and (c) contacting the potential mediator with GSK3 to determine whether the potential mediator mediates the activity of GSK3.

In a further aspect, the invention provides a computer for producing a three-dimensional representation of a molecule or molecular complex, the molecule or molecular complex including a binding pocket defined by at least some of a GSK3 crystal's atomic coordinates, or a three-dimensional representation of a homologue of the molecule or molecular complex. In one embodiment, the computer includes (a) a machine-readable data storage medium comprising a data storage material encoded with machine-readable data, the data including the GSK3 crystal's atomic coordinates; (b) a working memory for storing instructions for processing the machine-readable data; (c) a central-processing unit coupled to the working memory and to the machine-readable data storage medium for processing the machine readable data into the three-dimensional representation; and (d) a display coupled to the central-processing unit for displaying the three-dimensional representation.

The invention also provides a computer for determining at least a portion of the atomic coordinates corresponding to an X-ray diffraction pattern of a molecule or molecular complex. In one embodiment, the computer includes (a) a machine-readable data storage medium comprising a data storage material encoded with machine-readable data, wherein the data includes at least a portion of a GSK3 crystal's atomic coordinates; (b) a machine-readable data storage medium comprising a data storage material encoded with machine-readable data, wherein the data includes an X-ray diffraction pattern of the molecule or molecular complex; (c) a working memory for storing instructions for processing the machine-readable data of (a) and (b); (d) a central-processing unit coupled to the working memory and to the machine-readable data storage medium of (a) and (b) for performing a Fourier transform of the machine readable data of (a) and for processing the machine readable data of (b) into structure coordinates; and (e) a display coupled to the central-processing unit for displaying the structure coordinates of the molecule or molecular complex.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is an illustration of the structure of the GSK3-β ternary complex;

FIGS. 2A and 2B are surface representations of the GSK3-β ternary complex active site with bound peptide;

FIGS. 3A-3C are illustrations of a representation of a compound soaked into the active site of GSK3-β;

FIG. 4 is a flow diagram of a representative method of the invention using the three-dimensional structure of the GSK3-β ternary complex for identifying possible therapeutic compounds for mediating GSK3-β activity; and

FIG. 5 is a flow diagram of a representative method of the invention using the three-dimensional structure of the GSK3-β ternary complex for identifying possible therapeutic compounds for mediating GSK3-β activity.

### DETAILED DESCRIPTION OF THE PREFERRED EMBODIMENT

In accordance with the present invention, crystals of a ternary complex that includes a protein construct of human glycogen synthase kinase 3-β (GSK3-β) containing the protein's catalytic kinase domain, adenosine diphosphate, and a phosphorylated peptide are provided. Methods for crystallizing the ternary complex, the three-dimensional structure of the ternary complex, and methods for using the three-dimensional structure of the ternary complex for the identification of possible therapeutic compounds in the treatment of various disease conditions mediated by GSK3-β activity are provided.

### The Ternary Complex: GSK-3 Construct, Adenosine Diphosphate, And Phosphorylated Peptide

The ternary complex of the invention includes a GSK3-β construct, adenosine diphosphate, and a phosphorylated peptide. The complex can be formed by combining a GSK3 construct with adenosine triphosphate (ATP) and a peptide that is capable of phosphorylation by the GSK-3 construct. Peptide phosphorylation provides the complex including a phosphorylated peptide and adenosine diphosphate (ADP). The complex can also be formed by combining a phosphorylated peptide and adenosine diphosphate with the GSK3-β construct.

As described below, the crystal structure of the ternary complex structure shows that the phosphorylated peptide (e.g., a diphosphorylated peptide) spans the area between two symmetry related proteins in the crystal and is positioned to accept another serine residue. Without being bound by the theory, it is believed that the phosphorylated peptide may catalyze crystal formation of the ternary complex.

Compared to other GSK3 crystals, the ternary complex crystals of the invention offer the advantages of stability, ease of soaking in candidate GSK mediators, and high resolution. For example, crystals grown by the method described in Characterization of the GSK-3β Protein and Methods of Use Thereof, U.S. Patent Application No. 60/233,538, filed Sep. 19, 2000, and PCT/US01/29549, filed Sep. 19, 2001, each expressly incorporated herein by reference in its entirety, provide crystals that have limitations with regard to resolution (2.2 Angstroms maximum), time of data collection (typically 36 hours), and the size of ligand which can soaked into the ATP-binding site (approximately less than about 400 daltons). The ternary complex crystals are superior in all aspects: the maximum resolution achieved is 2.0 Angstroms (with 1.8 Angstroms possible); the time of data collection is drastically reduced (typically 24 hours); and there is no identified limitation to the mass of ligand which can be soaked into the ATP-binding site.

Various candidate GSK3 mediators (i.e., lead compounds) can be soaked into crystals of the ternary complex and their crystal structures determined. In the resulting crystals, the lead compounds are soaked in and ADP and phosphorylated peptide soak out. Because of the nature of the ternary complex crystals, relatively large lead compounds can be accommodated by the crystal. This appears to be a result of the phosphorylated peptide on the crystal structure. The binding site of the enzyme in the ternary complex crystal appears not to be occluded by crystal packing. This is in contrast to other GSK3 crystals studied, which suffer from a limitation of the size and shape of lead compound that can be accommodated by the crystal. The crystals of the ternary complex afford relatively unobstructed access to the enzyme's binding site. As noted above, crystals prepared as described in PCT/US01/29549 cannot easily soak compounds of greater than about 400 daltons, while crystals of the present invention can easily soak compounds having molecular weights in excess of 450 daltons.

Crystals of the ternary complex crystals can be highly resolved (e.g., 1.8 Angstrom). Similarly, co-crystals including the enzyme and candidate mediator can also be highly resolved (e.g., 2.6 Angstrom or better).

As noted above, the ternary complex includes a phosphorylated peptide that can be produced by the action of ATP, magnesium, and the GSK3 construct. The phosphorylated peptide can be derived from a peptide that is capable of phosphorylation by the GSK3 construct. Suitable peptides that are capable of phosphorylation include amino acid residues that can be phosphorylated. Suitable amino acid residues that can be phosphorylated include serine and threonine, among others. The phosphorylated peptide can also be derived from a phosphorylated peptide that includes an amino acid residue that can be phosphorylated (e.g., a monophosphorylated peptide can be phosphorylated to provide a diphosphorylated peptide). Other suitable peptides include, for example, diphosphorylated peptides that can be combined with the GSK3 construct to provide the complex. Suitable phosphorylated polypeptides include those that span the area between two symmetry related proteins in the crystal to provide relatively unobstructed access to the enzyme's binding site. Suitable peptides can include from about 6 to about 8 amino acid residues. In one embodiment, the phosphorylated peptide useful in combining with ATP and the GSK3 construct to provide the complex includes seven (7) amino acid residues including a phosphoserine residue. In another embodiment, the phosphorylated peptide useful in combining with ATP and the GSK3 construct to provide the complex includes six (6) amino acid residues including a phosphoserine residue. A representative phosphorylated peptide that can be combined with ATP and the GSK3 construct to provide the ternary complex has the sequence: LSRRPS*Y (SEQ ID NO: 2), where S* represents a phosphoserine. It will be appreciated that other phosphorylated peptides, such as diphosphorylated peptides, and other peptides, such a peptides that can be phosphorylated, can be used to provide the ternary complex of the invention.

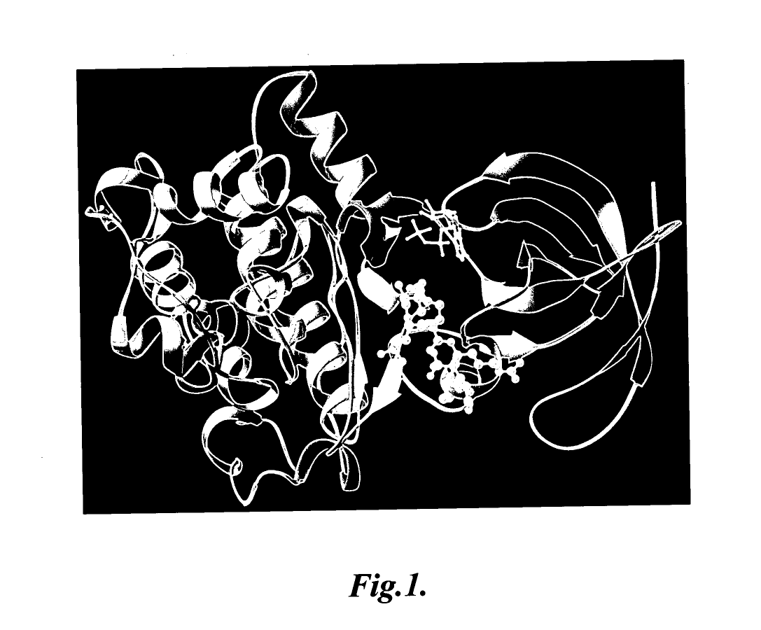

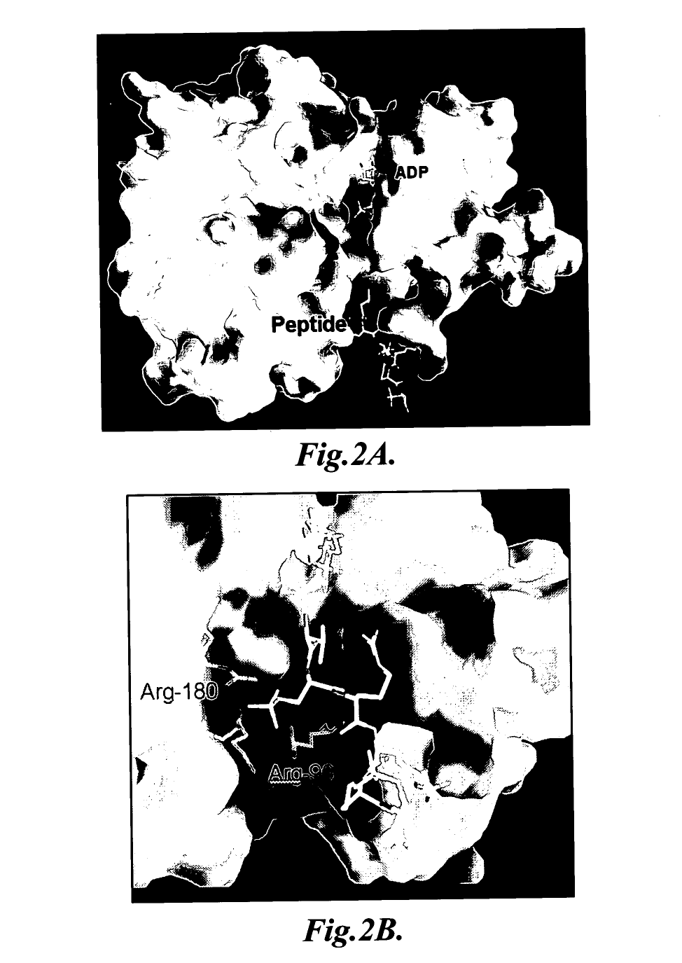

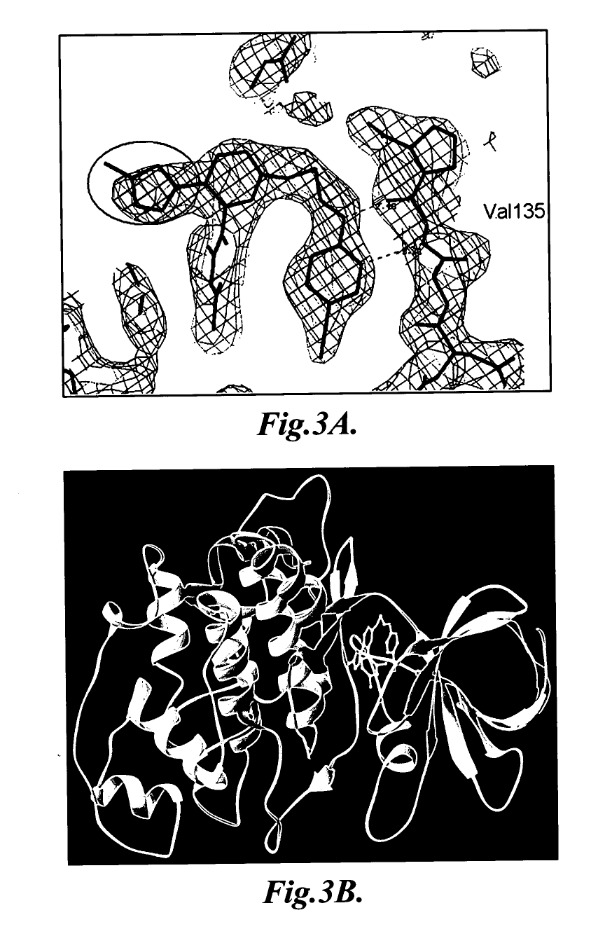

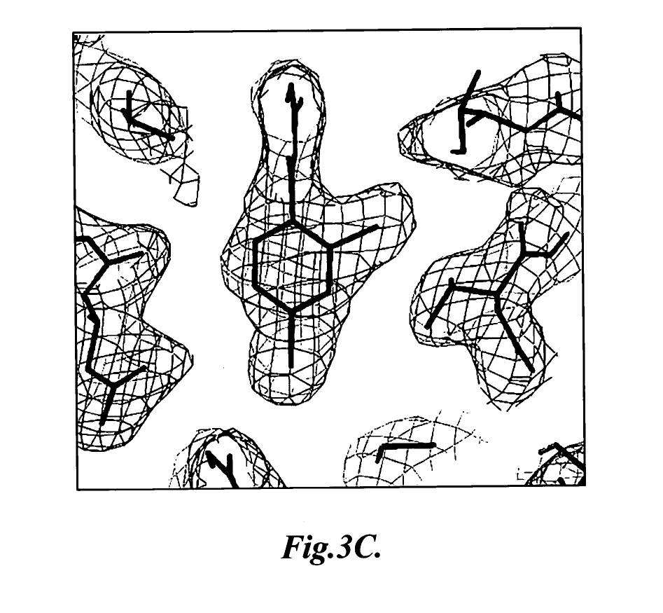

FIG. 1 is an illustration of the structure of a GSK 3-β ternary complex showing a polypeptide bridging the enzyme. FIGS. 2A and 2B are surface representations of a GSK 3-β ternary complex active site with bound polypeptide. FIGS. 3A-C is an illustration of a representative compound soaked into the active site of a GSK 3-β ternary complex. The compound: 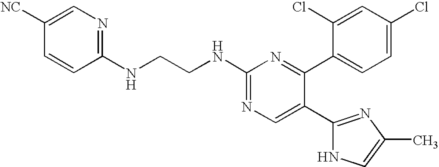
is shown interacting with the enzyme's active site: the compound's amino-pyridine group forms two hydrogen bonds to the linker region, the compound's imidazole forms no hydrogen bonds to the β-strand region, and the compound's dichlorophenyl group fits into the hydrophobic pocket near the catalytic region of the kinase. The three-dimensional structure of the GSK3-β construct with the above compound soaked in provided as a tabulations of atomic coordinates is given in Table 3.

### The GSK3-β Protein Construct: Expression, Purification, And Crystallization

In one aspect, the invention provides a composition that is a ternary complex that includes a GSK3-β construct, adenosine diphosphate, and a phosphorylated peptide. The GSK3-β construct contains the protein's catalytic kinase domain. The construct includes at least residues 37-384 of human GSK3-β and lacks the 36 amino acids at the protein's C-terminus. The composition is a crystalline form sufficient for structure determination by diffraction studies by X-ray.

It will be appreciated that GSK3 protein constructs other than the construct described herein, for example, active mutants or variants thereof, can provide three- dimensional structural information useful in identifying possible therapeutic compounds in the treatment of various disease conditions mediated by GSK3 activity.

Construct Sequence. The construct sequence, SEQ ID NO: 1, is provided below. The asterisk indicates the first residue that is seen in the crystal structure. The following construct and additional useful constructs and their preparation are described in co-pending U.S. Patent Application No. 60/221,242, filed Jul. 27, 2000, the disclosure of which is incorporated herein by reference in its entirety and for all purposes.

(SEQ ID NO: 1)

N-terminus: MEYMPMEGGGGSK
*VTTVVATPGQGPDRPQEVSYTDTKVIGNGSFGVVYQAKLCDSGELVAIK
KVLQDKRFKNRELQIMRKLDHCNIVRLRYFFYSSGEKKDEVYLNLVLDYV
PETVYRVARHYSRAKQTLPVIYVKLYMYQLFRSLAYIHSFGICHRDIKPQ
NLLLDPDTAVLKLCDFGSAKQLVRGEPNVSYICSRYYRAPELIFGATDYT
SSIDVWSAGCVLAELLLGQPIFPGDSGVDQLVEIIKVLGTPTREQIREMN
PNYTEFKFPQIKAHPWTKVFRPRTPPEAIALCSRLLEYTPTARLTPLEAC
AHSFFDELRDPNVKLPNGRDTPALFNFTTQELSSNPPLATILIPPHAR
I: C-terminus

Construct Purification. The GSK3-β protein construct was extracted from SF-9 cells infected with a baculovirus carrying GSK3-β 580 cDNA construct. The GSK3-β protein construct was purified to apparent homogeneity using S-Fractogel, Phenyl-650 M, and Glu-tag affinity chromatographies. The purified protein was then concentrated for crystallization. Purification of the construct is described in Example 1.

Construct Crystallization. Protein crystals can be formed from solutions of the GSK3 construct by, for example, the hanging drop technique. A representative method for forming suitable crystals of the GSK3 construct suitable for structure determination is described in Example 2.

It will be appreciated that various crystallization methods including, for example, microcrystallization methods can be utilized to obtain three-dimensional structural information useful in identifying possible therapeutic compounds in the treatment of various disease conditions mediated by GSK3 activity.

### The GSK3-β Protein Construct Structure

In another aspect of the invention, the three-dimensional structure of the GSK3 ternary complex is provided. Amino acid sequence data and atomic coordinates derived from X-ray diffraction data were used to determine the construct's three-dimensional structure. The construct's atomic coordinates were calculated from an electron density map produced from the combination of X-ray diffraction and phase data.

With the GSK3 ternary complex available in crystalline form suitable for structural determination, the crystal structure can be obtained by a variety of techniques. In a representative method, diffraction patterns were obtained using an X-ray image plate device. Phase data was then obtained by molecular replacement. Electron density maps were then constructed and the structure solved and molecule built. The resulting structure was refined and the structure validated. The ultimate result was an atomic model of the GSK3 construct. A representative method for obtaining the GSK3 crystal structure is described in Example 3.

It will be appreciated that the GSK3 structure can be solved by a variety of methods.

The statistics for collecting the crystallographic data are summarized in Table 1.

TABLE 1
Data and Model Statistics for Structure Solution.

| | Native 2.0 Å |
|---|---|
| Space Group | P2(1) |
| Highest Resolution (Å) | 2.0 |
| R_{merge} (%) | 7.6 |
| I/sigmaσ(total) | 17.8 |
| Final Shell | 2.06-2.0 |
| I/sigmaσ(Final Shell) | 2.8 |
| R-factor (%) | 25.1 |
| Free-R factor (%) | 30.2 |

The three-dimensional structure of the GSK3-β ternary complex provided as a tabulation of atomic coordinates is given in Table 2. In the table, “OH2” refers to structural water molecules, and “TER” refers to the terminus of a peptide chain.

The three-dimensional structure of the GSK3-β ternary complex based on the derived crystal structure is schematically illustrated in FIG. 1. The ternary complex includes N-terminal and C-terminal domains with the active site formed between the two domains. The N-terminal domain includes a β-barrel. The active site region includes the ATP binding site, the magnesium binding/catalytic base site, and substrate binding site.

The three-dimensional structure of the GSK3-β ternary complex's active site (including the catalytic site and substrate binding site) based on the derived crystal structure is schematically illustrated in FIGS. 2A and 2B. The active site includes Pro136 and Phe67 among other amino acid residues.

The area of interaction of the phosphorylated peptide lies in the substrate binding region of the enzyme, not in the ATP binding site (the site of action of most of the lead drug compounds). It is believed that the interactions formed between the peptide and the enzyme, as well as the between the peptide and symmetry mates of the enzyme in the crystal matrix, are what allow a superior form of crystal to be formed. The interactions of the peptide with the enzyme are varied. The first set of key interactions are that the N-terminal phosphoserine of the phosphorylated peptide interacts with Arg-96 (an electrostatic interaction) and the main chain amide of Val-214 through a hydrogen bond. The second set of key interactions is that the second arginine of the peptide (proceeding towards the C-terminus) forms key electrostatic interactions with the adjacent symmetry mate of the enzyme by interacting with the main chain carbonyl of Pro-258′ and the side chain Asp-260′ (prime denotes symmetry mate). The peptide makes several other minor hydrophobic interactions with both the enzyme and adjacent symmetry mates.

Structural information of the apoprotein active site can provide a basis for the rational design of ligands leading to therapeutic compounds effective in the treatment of various disease conditions mediated by GSK3-β activity. Thus, the structural information obtained from the crystallographic data can be used to develop a ligand profile and for the rational design of drugs for mediating GSK3-β activity as described below.

GSK3 Structural Representation. As noted above, in one aspect, the invention provides a method for identifying possible therapeutic compounds in the treatment of various disease conditions mediated by GSK3-β activity. The method involves the use of a three-dimensional structural representation of the GSK ternary complex. The three-dimensional structural representation may be a representation that includes (a) the complete GSK construct, (b) a fragment of GSK3 that includes the GSK construct, or (c) a fragment of the GSK construct that includes the amino acids that interact with ligands that can mediate GSK3 activity.

The structural representation is preferably based on or derived from the atomic coordinates as set out in Table 2, which represents the structure of the complete GSK construct. Suitable structural representations include three-dimensional models and molecular surfaces derived from these atomic coordinates. The coordinates in Table 2 include structural water molecules. These will vary, and may even be absent, in other models derived structurally (they are resolution and space group dependent). These solvent molecules will vary from crystal to crystal.

Variants of the atomic coordinates noted in Table 2 can also be used for the invention, such as variants in which the RMS deviation of the x, y, and z coordinates for all heavy (i.e., not hydrogen) atoms are less than about 2.5 Å, for example, less than about 2 Å, preferably less than about 1 Å, more preferably less than about 0.5 Å, or most preferably less than about 0.1 Å) compared with the atomic coordinates noted in Table 2. Coordinate transformations that retain the three-dimensional spatial relationships of atoms can also be used to give suitable variants.

The atomic coordinates provided herein can also be used as the basis of models of further protein structures. For example, a homology model could be based on the GSK construct structure. The coordinates can also be used in the solution or refinement of further crystal structures of GSK3, such as co-crystal structures with new ligands.

GSK3 Structural Representation Storage Medium. The atomic coordinates of the GSK ternary complex can be stored on a medium for subsequent use with a computational device, such as a computer (e.g., supercomputer, mainframe, minicomputer, or microprocessor). Typically, the coordinates are stored on a medium useful to hold large amounts of data, such as magnetic or optical media (e.g., floppy disks, hard disks, compact disks, magneto-optical media (“floptical” disks, or magnetic tape) or electronic media (e.g., random-access memory (RAM), or read-only memory (ROM). The storage medium can be local to the computer, or can be remote (e.g., a networked storage medium, including the Internet). The choice of computer, storage medium, networking, and other devices or techniques will be familiar to those of skill in the structural/computational chemistry arts.

The invention also provides a computer-readable medium for a computer, which contains atomic coordinates and/or a three-dimensional structural representation of the GSK ternary complex. The atomic coordinates are preferably those noted in Table 2 or variants thereof. Any suitable computer can be used in the present invention.

GSK3-β Ligand Profile Development. As noted above, the structural information obtained from the crystallographic data can be used to develop a ligand profile useful for the rational design of compounds for mediating GSK3-β activity. A ligand profile can be developed by taking into account the structural information obtained as described above for the apoprotein. The ligand profile can be further developed and refined with the determination of additional structures of protein with bound ligands. The ultimately developed ligand profile identifies possible therapeutic compounds for mediating GSK3-β activity.

The ligand profile can be primarily based on a shape interaction between the ligand and the protein ligand binding site. The evaluation of the shape interaction can include consideration of the ligand's conformational properties, ranking ligands based on their ability to achieve low energy conformations compatible with the ligand binding site. The shape interaction can also seek to maximize enthalpic interactions between the ligand and the binding site.

The process of developing a ligand profile can vary widely. For example, the profile can be developed by visual inspection of active site structures by experts. Such an inspection can include the consideration of the binding site and ligand structures and compound database searching. The development of the profile can also consider biological data and structure activity relationships (SAR) as well the consideration of known ligand binding interaction with other similar proteins.

In any event, the ligand profile is developed by considering ligand binding interactions including primary and secondary interactions and results in defining the pharmacophore. The term “pharmacophore” refers to a collection of chemical features and three-dimensional constraints that represent specific characteristics responsible for a ligand's activity. The pharmacophore includes surface-accessible features, hydrogen bond donors and acceptors, charged/ionizable groups, and/or hydrophobic patches, among other features.

In addition to the process for ligand profile development noted above, other structure-based drug design techniques can be applied to the structural representation of the GSK3 construct in order to identify compounds that interact with GSK3 to mediate GSK3 activity. A variety of suitable techniques are available to one of ordinary skill in the art.

Software packages for implementing molecular modeling techniques for use in structure-based drug design include SYBYL (available from Tripos Inc.); AMBER (available from Oxford Molecular); CERIUS² (available from Molecular Simulations Inc.; INSIGHT II (available from Molecular Simulations Inc.); CATALYST (available from Molecular Simulations Inc.); QUANTA (available from Molecular Simulations Inc.); HYPERCHEM (available from Hypercube Inc.); FIRST DISCOVERY (available from Schrodinger Inc.), MOE (available from Chemical Computing Group), and CHEMSITE (available from Pyramid Learning), among others.

The modeling software can be used to determine GSK3 binding surfaces and to reveal features such as van der Waals contacts, electrostatic interactions, and/or hydrogen bonding opportunities. These binding surfaces can be used to model docking of ligands with GSK3, to arrive at pharmacophore hypotheses, and to design possible therapeutic compounds de novo.

### GSK3-β Ligand Virtual Screening

The three-dimensional structure of the apoprotein, and the structure of the protein's active site in particular, allows for the determination of the fit of compounds into the active site. Utilizing a fast docking program, individual compounds from, for example, a compound database, can be evaluated for active site binding. The fit of a particular compound can be evaluated and scored. Setting a score threshold can then provides a family of compounds as a solution to the virtual screen.

At the first level, the virtual screen takes into account the three-dimensional structure of the apoprotein's active site. At the second level, the virtual screen considers the ligand profile and can utilize information obtained from the determination of the structure of protein with bound ligand. A virtual screen is possible even if there is no structural information on a bound ligand.

Information gained from the virtual screen can be considered to further develop the ligand profile. Alternatively, where the results of the virtual screen indicate a promising compound, the compound can be obtained and screened for the relevant biological activity.

Docking. Docking refers to a process in which two or more molecules are aligned based on energy considerations. Docking aligns the three-dimensional structures of two or more molecules to predict the conformation of a complex formed from the molecules (see, e.g., Blaney & Dixon, Perspectives in Drug Discovery and Design 1:301, 1993). In the practice of the present invention, molecules are docked with the GSK3 construct structure to assess their ability to interact with GSK3.

Docking can be accomplished by either geometric matching of the ligand and its receptor or by minimizing the energy of interaction. Geometric matching algorithms are preferred because of their relative speed.

Suitable docking algorithms include DOCK (Kuntz et al., J. Mol. Biol. 161:269-288, 1982, available from UCSF), the prototypical program for structure-based drug design; AUTODOCK (Goodsell & Olson, Proteins: Structure, Function and Genetics 8:195-202, 1990 and available from Oxford Molecular), which docks ligands in a flexible manner to receptors using grid-based Monte Carlo simulated annealing. The flexible nature of the AUTODOCK procedure helps to avoid bias (e.g., in orientation and conformation of the ligand in the active site) introduced by the user searcher (Meyer et al., Persp. Drug Disc. 3:168-95, 1995) because, while the starting conformation in a rigid docking is normally biased towards a minimum energy conformation of the ligand, the binding conformation may be of relatively high conformational energy (Nicklaus et al., Bioorganic & Medicinal Chemistry 3:411, 1995).

Other suitable docking algorithms include MOE-DOCK (available from Chemical Computing Group Inc.), in which a simulated annealing search algorithm is used to flexibly dock ligands and a grid-based energy evaluation is used to score docked conformations; FLExX (available from Tripos Inc.), which docks conformationally flexible ligands into a binding site using an incremental construction algorithm that builds the ligand in the site, and scores docked conformations based on the strength of ligand-receptor interactions; GOLD (Jones et al., J. Mol. Biol. 267:727-748, 1997), a genetic algorithm for flexible ligand docking, with full ligand and partial protein flexibility, and in which energy functions are partly based on conformation and non-bonded contact information; AFFINITY (available from Molecular Simulations Inc.), which uses a two step process to dock ligands: first, initial placements of the ligand within the receptor are made using a Monte Carlo-type procedure to search both conformational and Cartesian space; and second, a simulated annealing phase optimizes the location of each ligand placement, during this phase, AFFINITY holds the “bulk” of the receptor (atoms not in the binding site) rigid, while the binding site atoms and ligand atoms are movable; C² LigandFit (available from Molecular Simulations Inc.), which uses the energy of the ligand-receptor complex to automatically find best binding modes and stochastic conformation search techniques, with the best results from the conformational sampling retained. A grid method is used to evaluate non-bonded interactions between the rigid receptor and the flexible ligand atoms. DOCKIT (available from Metaphorics LLC) uses distance geometry for fast flexible ligand docking. GLIDE (available from Schrodinger Inc.) uses a pre-computed energy grid and an efficiently pruned systematic search for flexible docking.

Preferably, the docking algorithm is used in a high-throughput mode, in which members of large structural libraries of potential ligands are screened against the receptor structure (Martin, J. Med. Chem. 35:2145-54, 1992).

Suitable structural libraries include the ACD (Available Chemical Directory, form MDL Inc.), AsInEx, Bionet, ComGenex, the Derwent World Drug Index (WDI), the Contact Service Company database, LaboTest, ChemBridge Express Pick, ChemStar, BioByteMasterFile, Orion, SALOR, TRIAD, ILIAD, the National Cancer Institute database (NCl), and the Aldrich, Fluka, Sigma, and Maybridge catalogs. These are commercially available (e.g., the HTS Chemicals collection from Oxford Molecular, or the LeadQuest™ files from Tripos).

Defining the Pharmacophore. As noted above, a pharmacophore can be defined for the GSK3 ternary complex that includes surface-accessible features, hydrogen bond donors and acceptors, charged/ionizable groups, and/or hydrophobic patches, among other features. These features can be weighted depending on their relative importance in conferring activity (see, e.g., Computer-Assisted Lead Finding and Optimization, Testra & Folkers, 1997).

Pharmacophores can be determined using software such as CATALYST (including HypoGen or HipHop, available from Molecular Simulations Inc.), CERIUS2, or constructed by hand from a known conformation of a lead compound. The pharmacophore can be used to screen structural libraries, using a program such as CATALYST. The CLIX program (Davic & Lawrence, Proteins 12:31-41, 1992) can also be used, which searches for orientations of candidate molecules in structural databases that yield maximum spatial coincidence with chemical groups which interact with the receptor. The DISCO program (available from Tripos) uses a method of clique detection to identify common pharmacophoric features in each structure, produce optimally aligned structures, and extract the key features of the pharmacophore. The GASP program (available from Tripos) uses a genetic algorithm to automatically find pharmacophores with conformational flexibility.

de novo Compound Design. The binding surface or pharmacophore of the GSK3 ternary complex can be used to map favorable interaction positions for functional groups (e.g., protons, hydroxyl groups, amine groups, acidic groups, hydrophobic groups and/or divalent cations) or small molecule fragments. Compounds can then be designed de novo in which the relevant functional groups are located in the correct spatial relationship to interact with GSK3.

Once functional groups or small molecule fragments which can interact with specific sites in the binding surface of GSK3 have been identified, they can be linked in a single compound using either bridging fragments with the correct size and geometry or frameworks which can support the functional groups at favorable orientations, thereby providing a compound according to the invention. While linking of functional groups in this way can be done manually, perhaps with the help of software such as QUANTA or SYBYL, automated or semi-automated de novo design approaches can also be used.

Suitable de novo design software includes MCDLNG (Gehlhaar et al., J. Med. Chem. 38:466-72, 1995), which fills a receptor binding site with a close-packed array of generic atoms and uses a Monte Carlo procedure to randomly vary atom types, positions, bonding arrangements and other properties; MCSS/HOOK (Caflish et al., J. Med. Chem. 36:2142-67, 1993; Eisen et al., Proteins: Str. Funct. Genet. 19:199-221, 1994; available from Molecular Simulations Inc.), which links multiple functional groups with molecular templates taken from a database; LUDI (Bohm, J. Comp. Aided Molec. Design 6:61-78, 1992, available from Molecular Simulations Inc.), which computes the points of interaction that would ideally be fulfilled by a ligand, places fragments in the binding site based on their ability to interact with the receptor, and then connects them to produce a ligand; GROW (Moon and Howe, Proteins: Str. Funct. Genet. 11:314-328, 1991), which starts with an initial “seed” fragment (placed manually or automatically) and grows the ligand outwards; SPROUT which includes molecules to identify favorable hydrogen bonding and hydrophobic regions within a binding pocket (HIPPO module), selects functional groups and positions them at target sites to form starting fragments for structure generation (EleFanT), generates skeletons that satisfy the steric constraints of the binding pocket by growing spacer fragments onto the start fragments and then connecting the resulting part skeletons (SPIDeR), substitutes hetero atoms into the skeletons to generate molecules with the electrostatic properties that are complementary to those of the receptor site (MARABOU), and the solutions can be clustered and scored using the ALLigaTOR module; LEAPFROG (available from Tripos Inc.), which evaluates ligands by making small stepwise structural changes and rapidly evaluating the binding energy of the new compound, keeps or discards changes based on the altered binding energy, and evolves structures to increase the interaction energy with the receptor; GROUPBUILD (Rorstein et al., J. Med. Chem. 36:1700, 1993), which uses a library of common organic templates and a complete empirical force field description of the non-bonding interactions between a ligand and receptor to construct ligands that have chemically reasonable structure and have steric and electrostatic properties complimentary to the receptor binding site; CAVEAT (Lauri and Bartlett, Comp. Aided Mol. Design 8:51-66, 1994), which designs linking units to constrain acyclic molecules; and RASSE (Lai, J. Chem. Inf. Comput. Sci. 36:1187-1194, 1996).

### GSK3-β Ligands

Most lead compounds that initiate structure-based design cycles are identified by high-throughput screening. As a result of high throughput screening and the ligand profile and virtual screening described above, ligands are identified having the requisite conformational energies to assume a suitable shape and bind with the protein's active site. In addition to having low conformational energy and spatial compatibility with the apoprotein active site, the identified ligands are preferably synthetically accessible. The identified ligands can then be obtained (e.g., commercially obtained or synthesized) and screened for biological activity. The identified ligands can also be co-crystallized with the protein construct and the three-dimensional structure determined for the protein with bound ligand. The information obtained from structure of the protein with bound ligand can then be used to further develop the ligand profile as described above.

Suitable GSK3-β biological screening methods for evaluating ligand biological activity are known and include, for example, those noted in U.S. Patent Application No. 60/193,043, filed Mar. 29, 2000, and expressly incorporated herein be reference in its entirety.

### Method For Rational Drug Discovery Using GSK3 Crystal Structures

In another aspect, the invention provides a method for using a GSK3 crystal structure, specifically the three-dimensional structure of the GSK3 construct's active site, to design ligands for binding to and mediating the activity of GSK3-β.

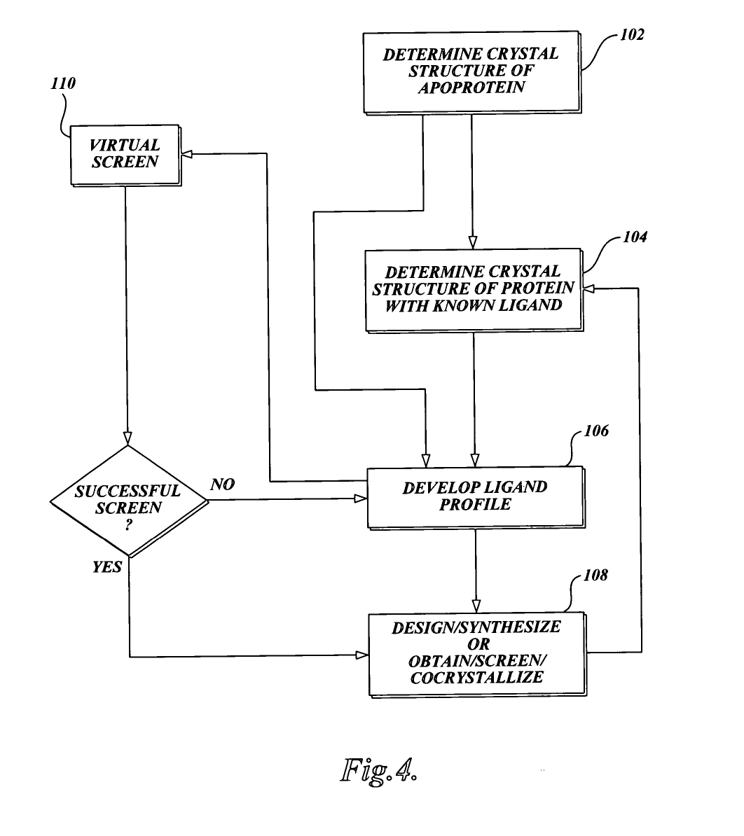

In one embodiment, the method is an iterative structure-based method for therapeutic compound design. A representative method is depicted by the flow diagram shown in FIG. 4. Referring to FIG. 4, the crystal structures of the apoprotein and the protein with bound ligand are determined in steps 102 and 104, respectively. From the structural information obtained from steps 102 and 104, a ligand profile is developed in step 106. A ligand profile can also be developed directly from the crystal structure of the apoprotein. Using the resulting profile, new ligands can be designed and/or obtained, screened for biological activity, and/or co-crystallized with the protein in step 108, or alternatively, the ligand profile can be used in a virtual screen in step 110. If the ligand obtained from the developed profile is co-crystallized, the structure of the co-crystal is determined in step 104 and the resulting structural information is used to further develop the ligand profile in step 106. If the ligand profile is used in a virtual screen in step 110, the virtual screen is either successful and identifies one or more ligands that can be obtained, screened, and/or co-crystallized in step 108. If the virtual screen is unsuccessful in identifying a suitable ligand, the ligand profile is further developed in step 106.

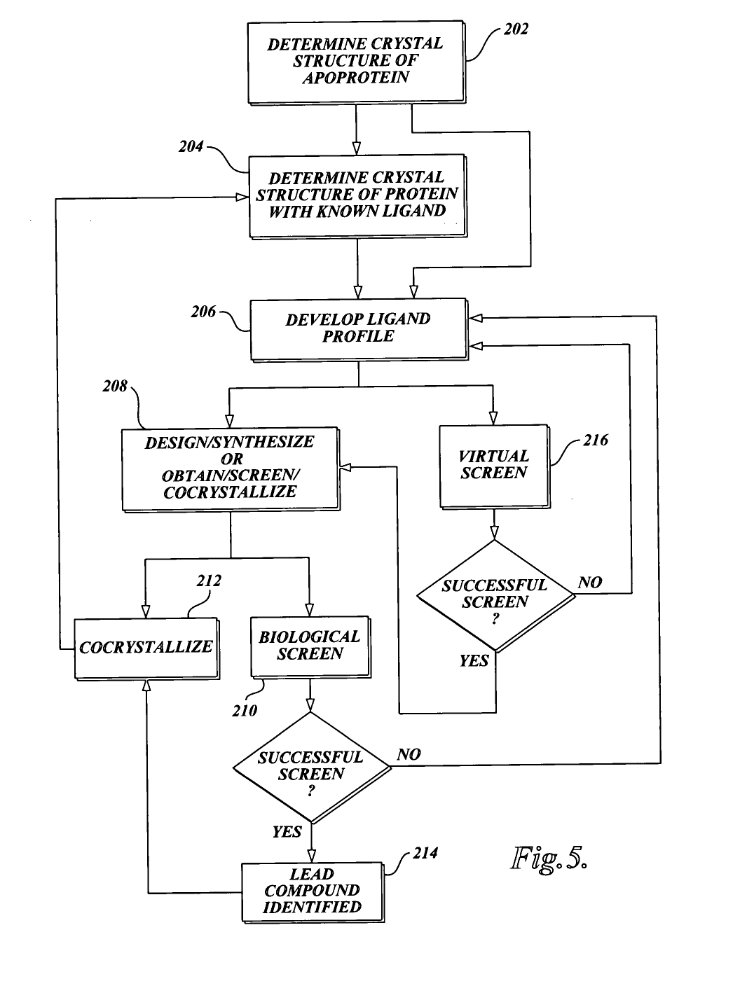

Lead compounds can be identified from biological screening of ligands developed by the ligand profile. A representative method for identifying a lead compound is depicted by the flow diagram shown FIG. 5. Referring to FIG. 5, the crystal structures of the apoprotein and the protein with bound ligand are determined in steps 202 and 204, respectively. From the structural information obtained from steps 202 and 204, a ligand profile is developed in step 206. A ligand profile can also be developed directly from the crystal structure of the apoprotein. From the resulting profile, a new ligand can be designed and/or obtained in step 208, and either screened for biological activity in step 210 and/or co-crystallized with the protein in step 212. If the biological screen is successful, a lead compound is identified in step 214. In a subsequent iteration, the lead compound can be co-crystallized in step 212 and iterations continued until a new drug candidate is identified. If the biological screen is not successful, that information can be used to further develop the ligand profile in step 206. If the ligand is co-crystallized, the co-crystal structure can be determined in step 204 and the structural information used in further developing the ligand profile in step 206.

Alternatively, the results of the ligand profile can be used in a virtual screen in step 216. If the virtual screen is successful and identifies one or more ligands, the ligand can be obtained in step 208 and screened in step 210 to determine its biological activity and whether or not a lead compound has been identified. The ligand obtained in step 208 can also be co-crystallized in step 212 and its structure determined and the resulting information used to further develop the ligand profile. If the virtual screen is unsuccessful in identifying a suitable ligand, the ligand profile is further developed in step 206.

### GSK3 Ligands And Their Uses

The method of the invention identifies ligands that can interact with GSK3. These compounds can be designed de novo, can be known compounds, or can be based on known compounds. The compounds can be useful pharmaceuticals themselves, or can be prototypes that can be used for further pharmaceutical refinement (i.e., lead compounds) in order to improve binding affinity or other pharmacologically important features (e.g., bio-availability, toxicology, metabolism, pharmacokinetics).

Accordingly, in another aspect, the invention provides (1) a compound identified using the method of the invention; (2) a compound identified using the method of the invention for use as a pharmaceutical; (3) the use of a compound identified using the method of the invention in the manufacture of a medicament for mediating GSK3 activity; and (4) a method of treating a patient afflicted with a condition mediated by GSK3 activity that includes administering an amount of a compound identified using the method of the invention that is effective to mediate GSK3 activity.

These compounds preferably interact with GSK3 with a binding constraint in the micromolar or, more preferably, nanomolar range or stronger.

As well as being useful compounds individually, ligands identified by the structure-based design techniques can also be used to suggest libraries of compounds for traditional in vitro or in vivo screening methods. Important pharmaceutical motifs in the ligands can be identified and mimicked in compound libraries (e.g., combinatorial libraries) for screening for GSK3-binding activity.

The foregoing and other aspects of the invention may be better understood in connection with the following representative examples.

### EXAMPLES

### Example 1

### GSK3-β Construct Purification

In this example, the purification of the GSK3-β protein construct is described. The construct was extracted from SF-9 cells infected with a baculovirus carrying GSK3-β 580 cDNA construct and purified to apparent homogeneity using S-Fractogel, Phenyl-650 M, and Glu-tag affinity chromatographies as described below.

Extraction. Cell paste from 20 L fermentation of infected SF-9 cells was washed 100 mL PBS (10 mM NaPi, pH 7.5, 150 mM NaCl) and then resuspended with 300 mL of Buffer H (20 mM Tris, pH 7.5, 1 mM tungstate, 1 mM arsenate, 50 mM DTT, 10 μg/mL leupeptin, 1 μg/mL pepstatin A, 10% glycerol, 0.35% octyl glycoside, 1 mM Mg²⁺). Cells were homogenized in a 100-mL Douncer (20 strokes with pestel B). The combined homogenate was centrifuged in a Ti45 rotor at 40,000 rpm for 35 minutes to remove cell debris and nuclei. The supernatant from the centrifugation were carefully decanted and filtered through 0.45μ filter.

S-Fractogel Chromatography. 175 mL S-fractogel (EM Science, Cat #18882) was packed into 5 cm×8.9 cm column and equilibrated with 5 column volumes of Buffer A (20 mM Tris, Ph 7.5, 10% glycerol). Prior to loading the filtered supernatant, one column volume of Buffer A containing 50 mM DTT was passed over the equilibrated column. The filtrate from the previous step was then loaded at 20 mL/min onto the column. The column was washed with 3 column volumes of Buffer A containing 50 mM DTT and 2 column volumes of Buffer A and then eluted with a linear gradient from 0 to 1 M NaCl in Buffer A over 20 column volumes. The eluant was fractionated into 20 mL fractions. Fractions containing GSK3 were detected by Western Blot using anti-GSK antibody (Santa Cruz Biotech, Cat #SC-7291). The Western-Blot positive fractions were pooled and mixed with equal volume of Buffer M (20 mM Tris, pH 7.5, 10% glycerol, 3.1 M NaCl) and filtered through a 0.45μ filter. The filtrate was used for Phenyl-650 M chromatography.

Phenyl-650 M Chromatography. 37.5 mL Phenyl-650 M (Tosohass, Cat #014943) was packed into a 2.2×10 cm column and equilibrated with 5 column volumes of Buffer C (20 mM Tris, pH 7.5, 10% glycerol, 1.6 M NaCl). Filtrate from S-fractogel step was loaded onto the column at 7.5 mL/min. After the loading was completed, the column was washed with 5 column volumes of Buffer C and eluted with linear gradient from 0% to 100% Buffer A (20 mM Tris, pH 7.5, 10% glycerol) over 20 column volumes. Fractions were collected at 15 mL each and GSK containing fractions were detected by Western Blot using anti-GSK antibody. The Western positive fractions were pooled and loaded onto a Glu-tag antibody affinity column.

Glu-tag Affinity Chromatography. 50 mg of Glu-tag antibody was immobilized onto 28 mL of Affi-Gel 10 (BioRAD, Cat #153-6046) and the packed into 2.2×6.5 cm column. The column was equilibrated with 5 column volumes of Buffer D (20 mM Tris, pH 7.5, 20% glycerol, 0.3 M NaCl, 0.2% octylglucoside) and the fraction pool from Phenyl-650 M step was loaded at 2.8 mL/min. After the loaded was completed, the column was wash with 5 column volumes of Buffer D and then eluted with 100 mL Glu-tag peptide (100 μg/mL) in Buffer D and fractionated into 4 mL fractions. GSK containing fractions were detected with SDS-PAGE and Coomassie Blue staining. These fractions were pooled, concentrated, and diafiltered into Buffer D to approximately 4.8 mg/mL in an Amicon concentrator using a 10 k MWCO YM10 membrane. The concentrated material was then submitted for crystallization.

### Example 2

### GSK3-β Construct Crystallization

In this example, the crystallization of the GSK3-β ternary complex is described.

A solution containing 100 mM of a seven residue peptide (N-LSRRPS*Y-C (SEQ ID NO: 3), purchased from Research Genetics), 20 mM ATP, 100 mM MgCl₂, and 100 mM Tris-HCl, pH 7.5 was mixed in a 1:10 ratio (v:v) with GSK-β protein solution in standard storage buffer. The GSK3-β protein solution used contained GSK3-β protein at 4.8 mg/ml in a storage buffer of 1×TBS, 300mM NaCl, 20% glycerol, 0.2% (v:v) octylglucopyranoside, and 5mM DTT. The resulting solution was incubated in an ice bucket at 4° C. for two hours. Following this incubation period, crystal drops were set up using the hanging drop method. A two-dimensional grid of a precipitant solution containing 7-12% (w:v) PEG 6000 and 5-8% MPD (v:v) with 100 mM HEPES, pH 7.5, as the buffer, was established in the reservoirs of a linbro culture plate. The protein solution (2 uL) was mixed with 2 uL of the precipitant solution from the reservoir on a glass cover slip. The cover slip was then placed over the reservoir of the well. Ternary crystals grew overnight and reached maximum size in four days. The crystals were cryopreserved in the standard GSK3-β cryosolution. The resulting crystals grow in the P21 space group with a monomer in the asymmetric unit and have the following approximate unit cell: a=57.0 Å, b=64.8 Å, c=57.2 Å, α=γ=90°, β=100.9°. These crystals diffract to better than 2.2 Å on an in-house X-ray source and to better than 2.0 Å on a synchrotron beamline. It should be noted that the peptide that actually appears to form the ternary complex is the diphosphorylated peptide, which must be formed in the enzymatic reaction during the incubation period.

Soaking of compounds for drug discovery is easily done by transplanting a crystal to a drop consisting of 5 uL of well solution (see above) and 1mM (or other appropriate concentration) of lead compound for 12-24 hours. During this time, the ADP and peptide ‘soak’ out of the crystal while the lead compound ‘soaks’ in. This lowers the resolution that the crystal is capable of diffracting to somewhat (to around 2.5 Angstroms), but allows rapid determination of the structure of the complex. These crystals seem impervious to changes caused by the compounds soaked in.

The crystals can be cryoprotected for data collection in a cryosolution consisting of 12% PEG 6000, 11% MPD, 0.1 M HEPES pH 7.5, 20% glycerol. The cryosolution can include from about 10 to about 14 percent by weight polyethylene glycol (PEG 6000), from about 9 to about 13 percent by weight 2-methyl-2,4-pentanediol (MPD), and from about 18 to about 22 percent by weight glycerol. The cryosolution can have a pH of from about 7.3 to about 7.7.

### Example 3

### GSK3-β Ternary Complex Crystal Structure Resolution

In this example, a representative method for resolving the crystal structure of the GSK3-β ternary complex is described.

The crystal structure of the GSK3-β ternary complex crystal structure was obtained using the C222(1) crystal structure (see PCT/JUS01/29549) and the program EPMR (Kissinger, et al., “Rapid Automated Molecular Replacement By Evolutionary Search, Acta Crystallogr D Biol Crystallogr. 55 (Pt 2):484-91, February 1999). The solution was then processed through several rounds of a refinement macrocycle. A typical refinement macrocycle consists of 100-200 rounds of conjugate gradient minimization, simulated annealing with either torsion or Cartesian dynamics, and grouped or individual temperature factor calculation. All refinement procedures were executed using the program CNX (Molecular Simulation, Inc.) This was followed by calculation of new electron density maps and manual rebuilding of the model based on features within these maps using the program O (DATAONO AB). All data from 50 Å-2.0 Å in the data set was used. It should be note that the majority of the structure is very similar to the crystal form of GSK3-β described in PCT/US01/29549. ADP and peptide were built into the electron density maps after the first round of refinement. The structure went through several rounds of refinement, including the addition of 235 structural waters, before the process converged at an R-factor of 25.1 and an R-free of 31.9 for all data from 50.0-2.0 Angstroms.

More traditional methods such as single/multiple isomorphous replacement or MAD phasing by themselves or in conjunction with molecular replacement could have been used equally as well. A description of these methods and other crystallographic principles can be found in Shoichet, B. K. and D. E. Bussiere, “The Role of Macromolecular Crystallography and Structure For Drug Discovery: Advances and Caveats, Current Opinion in Drug Discovery & Development 3 (4): 408-422, 2000.

The atomic coordinates for the GSK3-β ternary complex and the GSK3-β construct with representative compound soaked in are set forth in Tables 2 and 3, respectively. It will be appreciated that water positions will change from crystal to crystal.

LENGTHY TABLE REFERENCED HERE
US20070264699A1-20071115-T00001
Please refer to the end of the specification for access instructions.

LENGTHY TABLE REFERENCED HERE
US20070264699A1-20071115-T00002
Please refer to the end of the specification for access instructions.

While the preferred embodiment of the invention has been illustrated and described, it will be appreciated that various changes can be made therein without departing from the spirit and scope of the invention.

LENGTHY TABLE
The patent application contains a lengthy table section. A copy of the table is available in electronic form from the USPTO web site (http://seqdata.uspto.gov/?pageRequest=docDetail&DocID=US20070264699A1). An electronic copy of the table will also be available from the USPTO upon request and payment of the fee set forth in 37 CFR 1.19(b)(3).

The embodiments of the invention in which an exclusive property or privilege is claimed are defined as follows:

1. A crystallized GSK3-β complex, comprising:
(a) a GSK3 construct; and
(b) a phosphorylated polypeptide.

2. The complex of claim 1, wherein the construct has the amino acid sequence set forth in SEQ ID NO: 1 or an active mutant or variant thereof.

3. The complex of claim 1, wherein the phosphorylated polypeptide comprises a diphosphorylated polypeptide.

4. A crystallized GSK3-β complex, comprising:
(a) a GSK3-P construct having the atomic coordinates set forth in Table 2; and
(b) a phosphorylated polypeptide.

5. The complex of claim 4, wherein the phosphorylated polypeptide comprises a diphosphorylated polypeptide.

6. A polypeptide in a crystallized form, comprising the active form of GSK3 and the inhibitor binding site thereof, wherein the polypeptide comprises the atomic coordinates set forth in Table 2.

7. The polypeptide of claim 6 further comprising a bound ligand.

8. A method for crystallizing a human glycogen synthase kinase 3 (GSK3) protein, comprising:
crystallizing a purified GSK3 protein to provide a crystallized GSK3 protein having biological activity, wherein the crystallized GSK3 protein comprises a GSK3 construct and a phosphorylated polypeptide, and wherein the crystallized GSK3 protein is resolvable using x-ray crystallography to obtain x-ray patterns suitable for three-dimensional structure determination of the crystallized GSK3 protein.

9. The method of claim 8, wherein crystallizing the GSK3 protein comprises crystallizing by a hanging drop vapor diffusion method.

10. The method of claim 8, wherein the crystallized GSK3 protein comprises the atomic coordinates set forth in Table 2.

11. The method of claim 8, wherein the GSK3 protein comprises the amino acid sequence set forth in SEQ ID NO: 1 or an active mutant or variant thereof.

12. The method of claim 8, wherein the phosphorylated polypeptide comprises a diphosphorylated polypeptide.

13. A crystallized GSK3 protein provided by the method of claim 8.

14. A method for making a human glycogen synthase kinase 3 (GSK3) protein complex, comprising:
combining a polypeptide that is capable of being phosphorylated, adenosine triphosphate, a magnesium salt, and a GSK3 protein to provide a GSK3 protein complex comprising a phosphorylated polypeptide, adenosine diphosphate, and the GSK3 protein.

15. The method of claim 14, wherein the protein comprises the amino acid sequence set forth in SEQ ID NO: 1 or an active mutant or variant thereof.

16. The method of claim 14, wherein the polypeptide capable of being phosphorylated comprises a monophosphorylated polypeptide.

17. A method for making a human glycogen synthase kinase 3 (GSK3) protein complex, comprising:
combining a phosphorylated polypeptide, and a GSK3 protein to provide a GSK3 protein complex.

18. The method of claim 17, wherein the protein comprises the amino acid sequence set forth in SEQ ID NO: 1 or an active mutant or variant thereof.

19. The method of claim 17, wherein the phosphorylated polypeptide comprises a diphosphorylated polypeptide.

20. A method for making a human glycogen synthase kinase 3 (GSK3) protein crystal, comprising:
adding a precipitant to a solution comprising a polypeptide that is capable of being phosphorylated, adenosine triphosphate, a magnesium salt, and a GSK3 protein.

21. The method of claim 20, wherein the precipitant comprises polyethylene glycol.

22. The method of claim 20, wherein the precipitant comprises 2-methyl-2,4-pentanediol.

23. The method of claim 20, wherein the protein crystal comprises the atomic coordinates set forth in Table 2.

24. The method of claim 20, wherein the protein comprises the amino acid sequence set forth in SEQ ID NO: 1 or an active mutant or variant thereof.

25. The method of claim 20, wherein the phosphorylated polypeptide comprises a monophosphorylated polypeptide.

26. A crystallized GSK3 protein provided by the method of claim 20.

27. A method for making a human glycogen synthase kinase 3 (GSK3) protein crystal, comprising:
adding a precipitant to a solution comprising a phosphorylated polypeptide and a GSK3 protein.

28. The method of claim 27, wherein the precipitant comprises polyethylene glycol.

29. The method of- claim 27, wherein the precipitant comprises 2-methyl-2,4-pentanediol.

30. The method of claim 27, wherein the protein comprises the amino acid sequence set forth in SEQ ID NO: 1 or an active mutant or variant thereof.

31. The method of claim 27, wherein the phosphorylated polypeptide comprises a diphosphorylated polypeptide.

32. A method for making a human glycogen synthase kinase 3 (GSK3) protein crystal, comprising:
contacting a crystallized GSK3 protein with a potential GSK3 mediator, wherein the crystallized GSK3 protein comprises a GSK3 construct and a phosphorylated polypeptide.

33. The method of claim 32, wherein the GSK3 protein comprises the amino acid sequence set forth in SEQ ID NO: 1 or an active mutant or variant thereof.

34. The method of claim 32, wherein the phosphorylated polypeptide comprises a diphosphorylated polypeptide.

35. A crystallized GSK3 protein provided by the method of claim 32.

36. The complex of claim 1 having a P2(1) space group.

37. The complex of claim 1 having a unit cell a=57.0 Å, b=64.8 Å, c=57.2 Å, α=γ=90°, β=100.9°.


SEQUENCE LISTING (2 sequences)

SEQ ID NO: 1 - protein, 361 aa, Homo Sapiens
MEYMPMEGGGGSKVTTVVATPGQGPDRPQEVSYTDTKVIGNGSFGVVYQAKLCDSGELVAIKKVLQDKRFKNRELQIMRKLDHCNIVRLRYFFYSSGEKKDEVYLNLVLDYVPETVYRVARHYSRAKQTLPVIYVKLYMYQLFRSLAYIHSFGICHRDIKPQNLLLDPDTAVLKLCDFGSAKQLVRGEPNVSYICSRYYRAPELIFGATDYTSSIDVWSAGCVLAELLLGQPIFPGDSGVDQLVEIIKVLGTPTREQIREMNPNYTEFKFPQIKAHPWTKVFRPRTPPEAIALCSRLLEYTPTARLTPLEACAHSFFDELRDPNVKLPNGRDTPALFNFTTQELSSNPPLATILIPPHARI

SEQ ID NO: 2 - protein, 7 aa, Homo sapiens
  - (6)..(6): Xaa is a phosphoserine.
LSRRPXY